(19) United States
(12) Patent Application Publication
(10) Pub. No.: US 2004/0133054 A1
(43) Pub. Date: Jul. 8, 2004

(54) Dehydrogenation catalyst and process for preparing the same

(76) Inventors: Joseph E. Pelati, Charleston, WV (US); Robert J. Gulotty JR., Midland, MI (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/696,670

(22) Filed: Oct. 29, 2003

Related U.S. Application Data
(60) Provisional application No. 60/431,254, filed on Dec. 6, 2002.

(51) Int. Cl. B01J 23/72; C07C 4/06

(52) U.S. Cl. 585/444; 502/316; 502/324; 502/331

(57) ABSTRACT

A calcined dehydrogenation catalyst composition having iron and potassium supported on alumina has improved selectivity at a given hydrocarbon conversion by the addition of metal ions selected from indium, cerium, sodium, molybdenum and tungsten. A process for preparing the catalyst and for dehydrogenating alkylaromatic hydrocarbon compounds is also provided.

### CROSS REFERENCE STATEMENT

This application claims the benefit of U.S. Provisional Application No. 60/431,254, filed Dec. 6, 2002.

### BACKGROUND OF THE INVENTION

This invention relates to improved catalysts for the dehydrogenation of hydrocarbons and to a method of making such catalyst compositions which exhibit improved selectivity at a given conversion of the hydrocarbon starting material. The compositions are especially adapted for use in fluidized bed dehydrogenation processes.

It is known that processes and catalysts for dehydrogenating alkylaromatics, such as ethylbenzene, isopropylbenzene and butylene to produce styrene, cumene and butadiene, respectively, have been in use for decades. Catalytic dehydrogenation of hydrocarbons using various catalyst compositions has been known from just prior to World War II. Promoted iron oxide catalysts have been found to be especially useful in the dehydrogenation of alkyl aromatic hydrocarbons to vinyl aromatic hydrocarbons. Most commercial iron oxide dehydrogenation bulk catalysts include minor amounts of promoters, for example, salts or oxides of chromium, manganese, tungsten, cerium, sodium and molybdenum or bismuth, with chromium being preferred, together with a compound of potassium, for example, potassium oxide or carbonate. The potassium compound gives the catalyst a self-regenerative property that prolongs its useful life for long periods of time without significant loss of activity. Recent improvements include the incorporation of minor amounts of vanadium and modifiers, such as carbon black or graphite and methyl cellulose, which can beneficially affect the pore structures of the catalysts. Further, U.S. Pat. No. 5,376,613 teaches the inclusion of sodium or calcium as their oxides to improve moisture resistance and thereby add stability and improved crush strength. A class of dehydrogenation catalyst compositions containing red or yellow iron oxides and various catalyst promoters, as disclosed, for example, in U.S. Pat. Nos. 5,376,613; 3,703,593; and 4,684,619, all of which are assigned to The Dow Chemical Company, and are incorporated herein by reference, are known and have been used commercially.

In recent years, catalysts with higher amounts of potassium have been used. In U.S. Pat. No. 4,503,163 assigned to Mobil Oil Company, for example, catalysts are disclosed which contain 13-48 percent and preferably 27-41 percent by weight of a potassium promoter compound, calculated as potassium oxide. Such catalysts are self regenerative catalysts which perform well at lower steam to oil ratios; for example, ratios of <2:1 (by weight). The economic advantages of using less steam are obvious. The problem with using higher concentrations of potassium is that the vulnerability of the iron oxide catalyst to moisture increases with increasing potassium concentration.

Most recently a dehydrogenation catalyst using platinum, tin and an alumina support together with an element of the lanthanide group, such as lanthanum, were used to provide a fluidized bed catalyst for a process for dehydrogenating light paraffins to the corresponding light olefins, for example C₂-C₅, see U.S. Pat. No. 5,633,421. In European application EP 0 637 578 A1, published on Feb. 8, 1995, the inventors teach a process for preparing light olefins by reacting corresponding paraffins in a fluidized bed reactor in contact with a catalytic system containing gallium, platinum, and possibly one or more alkali or alkaline earth metals on an alumina support of a specified type. Further, in European published application EP 0 885 654 A1, published Dec. 23, 1998, a catalytic system for dehydrogenating ethylbenzene to styrene, containing chromium oxide, tin oxide, at least one oxide of an alkaline metal and an alumina carrier is taught, especially for use in a fluidized bed reactor and regenerator process. Still further in published PCT application WO 0123336 A1 20010405, while stating a broader range, employs an iron oxide catalyst, at a concentration of from 6.6-10.4 percent by weight and having a support with a packed bulk density greater than 1.45 g/ml. The catalyst is used in a fluid-bed reactor-regenerator for the dehydrogenation of ethylbenzene to styrene.

In co-pending application U.S. Ser. No. 01/02673, filed Jan. 24, 2001, assigned to the assignee of this invention, there is taught an integrated process for the preparation of styrene from ethane and benzene in which ethane and ethylbenzene are simultaneously dehydrogenated generally according to the process of Iezzi in published application EP 0 637 578 A1, referenced above, and the effluent produced contains ethylene, styrene and by-products, which are separated. The ethylene produced in the dehydrogenation reaction goes to an alkylation unit with fresh benzene feed which produces the ethylbenzene for dehydrogenation. Thus, the fresh feeds to the integrated dehydrogenation and alkylation are ethane and benzene and the primary product is styrene. The dehydrogenation catalyst used by Iezzi et al patent application WO 0123336 A1 is a gallium and platinum catalyst with possibly one or more alkaline or alkaline earth metals and a support consisting of certain phases of alumina.

It has now been found that the selectivity to styrene at a given ethylbenzene conversion can be improved when certain metal ions are added to the iron/potassium dehydrogenation catalysts. A further finding is that the alumina support having specifically defined bulk density and particle size characteristics or properties provides a preferred catalyst for use in fluidized bed reactors and regenerators. Still further, the combination of the catalyst and process of making it as presented in the present specification provides a more economical process for the preparation of vinyl aromatic hydrocarbons and diolefins, such as ethylbenzene, cumene, butadiene and similar products.

### BRIEF SUMMARY OF THE INVENTION

The present invention provides a novel catalyst composition for the dehydrogenation of paraffinic or alkylaromatic hydrocarbons. More particularly, there is provided a calcined dehydrogenation catalyst which comprises a calcination product of (a) at least one iron oxide or other iron compound; (b) a compound of an alkali metal; (c) a compound of a member of the group consisting of indium, calcium, samarium, cerium, sodium, molybdenum, tungsten, zinc, manganese, copper and lanthanum; and (d) an alumina or alumina-silica support material having specified characteristics which facilitate contact with the active catalytic sites and are capable of Geldart A fluidizable properties. Additionally, the present invention includes a process for preparing the calcined dehydrogenation catalyst which comprises the steps of (a) adding an active phase in the form of an aqueous solution or suspension of (i) at least one iron oxide, carbonate, bicarbonate, nitrate, hydroxide, oxalate or other similar conjugate base of a weak acid; (ii) a compound of an alkali metal; and (iii) a compound of a member of the group consisting of indium, calcium, samarium, cerium, sodium, molybdenum, tungsten, zinc, manganese, copper, and lanthanum to an alumina or alumina-silica support material having specified characteristics which facilitate contact with the active catalytic sites and are capable of Geldart A fluidizable properties; (b) drying the support material containing the active phase to remove the water; and (c) calcining the dried support material containing the active phase to a finished catalyst. The finished catalyst is more selective to styrene, cumene or butadiene, depending on the respective starting material, at a given conversion of the paraffinic or alkylaromatic hydrocarbon than comparable catalysts without the compound containing a member of the group consisting of indium, calcium, samarium, cerium, sodium, molybdenum, tungsten, zinc, manganese, copper, and lanthanum. The finished catalyst is effective in fluidized bed reactors and regenerators. As a result, the catalyst composition of the present invention provides a more economical process for the production of unsaturated or vinyl aromatic hydrocarbons, particularly styrene, cumene and butadiene.

### DETAILED DESCRIPTION OF THE INVENTION

As indicated hereinabove, the catalyst composition of the present invention features a calcined dehydrogenation catalyst comprising a calcination product of a) at least one iron oxide or a carbonate, bicarbonate, nitrate, hydroxide, oxalate or other similar conjugate base of a weak acid; b) a carbonate, bicarbonate, nitrate, hydroxide, oxide, oxalate, or other similar conjugate base of a weak acid of an alkali metal; c) a carbonate, bicarbonate, nitrate, hydroxide, oxide, oxalate or other similar conjugate base of a weak acid of at least one member of the group consisting of indium, calcium, samarium, cerium, sodium, molybdenum, tungsten, zinc, manganese, copper and lanthanum; and d) an alumina or silica-alumina support material having a bulk density from 0.9 to 1.3 grams per cubic centimeter, and an average particle size of from 30 to 300 microns. Preferred members of the group listed in (c) are indium, calcium, samarium, cerium and sodium with the most preferred being a calcined catalyst comprising the member of the group listed in (c) as indium.

The group (c) compounds may be termed selectivity improvers because they generally improve the selectivity of the converted paraffinic or alkylaromatic hydrocarbons to unsaturated compounds. Such paraffinic or alkylaromatic hydrocarbons which can be treated in the process of this invention using the novel catalysts of this invention are hydrocarbon compounds which are capable of being dehydrogenated and which generally range from C₂ to C₂₅ (that is they contain from 2 to 25 carbon atoms in the molecule). Preferred paraffinic hydrocarbon compounds are from C₂ to C₂ and most preferably from C₂ to C₉. Typically, hydrocarbons which are useful in the present invention are alkyl, alkaryl, or alkenyl hydrocarbons, such as ethane, propane, butane, pentane, hexane, heptane, octane and such paraffins which are hydrocarbyl substituted paraffins. Likewise, unsubstituted or substituted aryl compounds, such as, benzene, toluene, xylene, ethylbenzene, isopropylbenzene, and methyl ethylbenzene can be used as starting materials. The products are the dehydrogenated compounds corresponding to such starting paraffinic or alkylaromatic hydrocarbon compounds and have the same number of carbon atoms as the starting paraffinic or alkylaromatic hydrocarbon compound. Thus, ethylene, propylene, butylene, pentene, hexene, heptene, octene and their hydrocarbyl substituted analogs can be produced using the novel catalyst of this invention. Similarly, alkenyl substituted aryl compounds or vinyl aromatic hydrocarbon compounds, such as, styrene, cumene, and α-methyl styrene can be produced. Depending on the feed streams introduced more than one product can be produced at one time using this novel catalyst, that is, feeding ethane and ethylbenzene to a reactor containing the catalyst of this invention can produce both ethylene and styrene. Similarly, feeding isopropane and isopropylbenzene will produce isopropylene and cumene in the present process.

The catalyst of the present invention features a support material which is comprised of alumina or a silica-alumina, in which the silica can be up to 10 weight percent of the total support material. An alumina with any acceptable phase of alumina which does not interfere with the dehydrogenation process can be used. Although Boehmite or α-alumina can be advantageously employed, other phases of alumina can also be used, such as beta, delta, theta and mixtures of all of these. The packed bulk density of the support material is important for the proper pressure differential over the length of the reactor in which the catalyst is employed. Bulk density also influences or is influenced by the phase of the alumina and the porosity of the alumina support material. It has been determined that a packed bulk density, as determined by ASTM-D416-82 and similar methodologies, of from 0.90 to 1.30 g/cc and, preferably, from 0.95 to 1.1 g/cc, is sufficient to have acceptable pressure differential over typical reactors and to have a satisfactory porosity for the catalyst of this invention when used in dehydrogenation reactions.

It is known from U.S. Pat. No. 5,376,613, previously incorporated by reference, that iron oxide and various catalyst promoters may be used in dehydrogenation catalysts. In the present invention, iron is generally added to the catalyst composition as red iron oxide, Fe₂O₃, or yellow iron oxide, Fe₂O₃H₂O. Particularly suited are pigment grades of red and yellow iron oxides. Likewise, the catalyst promoter can be any material taught by the art, for example, an alkali metal compound(s) that is converted to an alkali metal oxide under calcination conditions. Potassium compounds are the preferred promoters. The promoter can be added to the catalyst in various forms. The alkali metal oxides, hydroxides, carbonates, and bicarbonates, and mixtures thereof are preferred, and potassium carbonate or a mixture of potassium carbonate with potassium oxide is most preferred.

The catalyst compositions of the present invention also comprises selectivity improvers selected from the group consisting of indium, calcium, samarium, cerium, sodium, molybdenum, tungsten, zinc, manganese, copper, and lanthanum to enhance selectivity. Such selectivity improvers can be added to the catalyst in the form of an oxide or in the form of other compounds that decompose upon calcination to form oxides, as for example, carbonate, bicarbonate, nitrate, hydroxide or oxalate or any combination thereof and the oxide itself is likewise useful.

Other known catalyst additives can be included in the catalysts of the invention, but are not essential. A chromium compound which can serve as a stabilizer for the active catalytic components is illustrative of an optional but preferred additive. Chromium compounds have previously been added to alkali-promoted iron oxide catalysts to extend their life. Chromium, as optionally used in the compositions of this invention, can be added to the catalyst in the form of a chromium oxide or in the form of chromium compounds which decompose upon calcination to form chromium oxides.

Other metal compounds that may be added as promoters include compounds of aluminum, vanadium, cadmium, magnesium, and nickel, providing they can be calcined to the corresponding metal oxide.

The catalyst of the present invention is prepared by modifying an alumina or silica-alumina support, as specified hereinabove, using the incipient wetness method, in which an aqueous solution or suspension of the active species and promoters are added to the support material. The solution or suspension is stirred to ensure good contact and then the aqueous phase is evaporated to dryness and the dried material is calcined at elevated temperatures to secure the active phase on the support. The aqueous solution or suspension of active catalytic metal species, promoters, and selectivity improvers can employ various counter-ions, such as, the nitrate, carbonate, bicarbonate, hydroxide, oxalate or even the oxide itself. Other similar conjugate bases of weak acids can be used as the counterion in a compound added to the solution or suspension. Preparation of such aqueous solutions or suspensions of the active metal compounds, promoter compounds, and selectivity improver compounds are known and generally available to the skilled artisan in the catalytic industry. For convenience, the aqueous solution of metal compounds can be added to the alumina at temperatures from room temperature to 80° C. and generally at atmospheric pressure. The support material is thoroughly wetted with the aqueous solution and then dried to remove water. Finally, the dry support containing the active metal compounds is calcined at temperatures up to 950° C. to convert the active species to the finished catalyst, which is typically the metal oxide. Therefore, the present invention also features a process for preparing a calcined dehydrogenation catalyst comprising:

a) adding an active phase in the form of an aqueous solution or suspension of

(i) at least one iron oxide or a carbonate, bicarbonate, nitrate, hydroxide, oxide, oxalate or other similar conjugate base of a weak acid

(ii) a carbonate, bicarbonate, nitrate, hydroxide, oxide or oxalate or other similar conjugate base of a weak acid of an alkali metal; and

(iii) a carbonate, bicarbonate, nitrate, hydroxide, oxide, oxalate or other similar conjugate base of a weak acid of at least one member of the group consisting of indium, calcium, samarium, cerium, sodium, molybdenum, tungsten, zinc, manganese, copper and lanthanum

to an alumina or silica-alumina support material having a bulk density from 0.9 to 1.3 grams per cubic centimeter and an average particle size of from 30 to 300 microns;

b) drying the support material containing the active phase to remove the water; and

c) calcining the dried support material containing the active phase to a finished catalyst.

In order to determine which promoters and selectivity improvers are useful and a part of the present invention, an experimental procedure was developed to evaluate the catalyst compositions. The procedure includes a laboratory evaluation in which the catalyst composition to be tested is conducted in a single vessel using reactor-regeneration cycles. In a preferred evaluation procedure, a one inch internal diameter, up-flow quartz reactor was used. The reactor is separated into two zones, a preheat zone on the bottom separated by a quartz frit from the reaction zone. A multi-zone heating mantle surrounds the reactor. A separate heated vessel is used to vaporize the ethylbenzene which is mixed with the diluent gas. The diluent gas may be nitrogen, helium, argon, methane, ethane, or propane. The total gas flows are based on the packed volume of the catalyst with volume and density measured by the method of ASTM D4164-82. The total gas flows used were in the range of 300-600 hr⁻¹ gas hourly space velocity (GHSV) calculated as liters gas/hour per liters catalyst, but could be up to as much as 20,000 hr⁻¹. Normalized gas volumes are used and are calculated at one atmosphere pressure and 0° C. The mole fraction of ethylbenzene typically used is 5-30 percent. The reaction temperatures range from 500-650° C. The length of the reaction cycle is 5-30 minutes, while the regeneration cycle can be up to one hour using gas streams with oxygen concentrations up to 20 mole percent and temperatures up to 80° C.

Using the above catalyst preparation and evaluation procedures, several catalysts according to the present invention were made and evaluated, as shown in the following Examples.

### EXAMPLES

### Example 1

A spherical alumina support (180 grams) primarily composed of alpha phase alumina and a BET (Brunauer-Emmett-Teller method of measuring surface area, described by S. Brunauer, P. H. Emmett and E. Teller, Journal of the American Chemical Society, 60, 309 (1938), incorporated herein by reference) surface area of 92 square meters/gram and a pore volume of 0.4 cubic centimeters/gram was dried at 170° C. This alumina support had 1.6 weight percent silica. An aqueous solution (72 milliliters final volume) was prepared with 29.5 grams of Fe(III) nitrate nonahydrate, 1.43 grams of In(III) pentahydrate, and 12.5 grams of potassium nitrate. The alumina was cooled to room temperature and the metal salt solution was slowly added with good mixing. Then 3 ml of additional water was used to rinse the solution beaker and this was added to the alumina. The impregnated support was kept for two hours at room temperature, then heated at 80° C. for two hours, and finally dried for 12 hours at 170° C. The catalyst was calcined at 740° C. for 4 hours. A red-orange powder was produced with a theoretical active phase concentration (wt/wt) of 3 percent Fe₂O₃, 3 percent K₂O, and 0.26 percent In₂O₃.

The catalyst was sieved between 100 and 400 mesh screens. An 83 ml sample (1.09 g/ml packed, bulk density) was loaded into a 1 inch internal diameter up-flow fluidized bed quartz reactor. The diluent gas was nitrogen with a flow rate of 0.443 L/min. at 0° C. and 1 atmosphere for the reaction cycle. The reactor was heated to 550° C. as monitored and controlled from an internal thermocouple placed in the middle of the catalyst. The ethylbenzene flow was 0.66 ml/min. and this stream was mixed with the diluent and vaporized prior to introduction to the reactor. The reaction segment was run for 10 minutes. Then a nitrogen purge flowed through the reactor for 15 minutes. The effluent stream was cooled and condensed in a liquid nitrogen trap and the residual gaseous products were captured in a gas sampling bag. The nitrogen feed is switched to air at the same flow rate after the reactor temperature was increased to 650° C. for the regeneration segment and maintained for 30 minutes. The reactor was cooled to the next reaction temperature and the reactor was purged with nitrogen. All of the regeneration effluent is collected in a gas sampling bag. This reaction/regeneration cycle is done 2 times as a break-in procedure for the catalyst. Data collection starts with the third cycle.

The samples are analyzed by gas chromatography. The liquid sample weight is measured and each gas sample volume is determined. These data are combined to calculate an overall conversion and product selectivity. The conversion is calculated as moles of ethylbenzene converted per moles of ethylbenzene fed to the reactor. The selectivity is defined as the moles of styrene produced per moles of ethylbenzene converted.

The reaction/regeneration cycle is repeated for three different reaction temperatures between 550 and 600° C. The selectivity at 50 percent conversion (defined as S50) is interpolated by second order polynomial regression analysis. The data is shown in Table 1.

TABLE 1

| Temperature, ° C. | Percent Conversion | Percent Selectivity to Styrene |
|---|---|---|
| 550 | 39.2 | 87.0 |
| 550 | 39.2 | 86.3 |
| 575 | 50.8 | 84.8 |
| 600 | 61.2 | 80.2 |

The value of S50 for Example 1 is 85 percent.

### Example 2

The procedure for Example 1 is used to produce a catalyst of this invention, except that the following changes are used in the active phase composition. The active phase impregnation solution contained 29.5 g Fe(III) nitrate nonahydrate, 12.5 g potassium nitrate, and 3.6 g of an aqueous cerium(IV) nitrate solution (assay=28 weight percent CeO₂). After calcination, a red-orange powder was produced with a theoretical active phase concentration (wt/wt) of 3 percent Fe₂O₃, 3 percent K₂O, and 0.50 percent CeO₂. The catalyst bulk density was 1.05 g/cc. The selectivity/conversion data are shown in Table 2.

TABLE 2

| Temperature, ° C. | Percent Conversion | Percent Selectivity to Styrene |
|---|---|---|
| 550 | 31.2 | 87.4 |
| 550 | 31.7 | 86.4 |
| 575 | 44.6 | 85.4 |
| 600 | 59.1 | 76.8 |

The S50 is determined by second order polynomial regression to be 83 percent

### Example 3

The procedure of this example is identical to Example 1, except that the active phase impregnation solution contained 29.5 g Fe(III) nitrate nonahydrate, 12.5 g potassium nitrate, 10 and 0.30 g of a 50 wt percent aqueous NaOH solution. After calcination, a red-orange powder was produced with a theoretical active phase concentration (wt/wt) of 3 percent Fe₂O₃, 3 percent K₂O, and 0.09 percent Na₂O. The packed bulk density of the catalyst was 1.07 g/cc. The selectivity/conversion data are shown in Table 3.

TABLE 3

| Temperature, ° C. | Percent Conversion | Percent Selectivity to Styrene |
|---|---|---|
| 550 | 33.2 | 87.2 |
| 550 | 34.8 | 87.8 |
| 575 | 44.8 | 85.3 |
| 600 | 59.4 | 78.3 |

The value of S50 interpolated by second order polynomial regression analysis for Example 3 is 83 percent

### Example 4

The procedure of this example is identical to Example 1, except that the active phase impregnation solution contained 29.5 g Fe(III) nitrate nonahydrate, 12.5 g potassium nitrate, and 1.2 g. ammonium heptamolybdate tetrahydrate. After calcination, a red-orange powder was produced with a theoretical active phase concentration (wt/wt) of 3 percent Fe₂O₃, 3 percent K₂O, and 0.51 percent MoO₃. The catalyst packed, bulk density was 1.08 g/cc. The selectivity/conversion data are shown in Table 4.

TABLE 4

| Temperature ° C. | Percent Conversion | Percent Selectivity to Styrene |
|---|---|---|
| 550 | 33.3 | 85.7 |
| 575 | 44.5 | 83.6 |
| 600 | 60.8 | 79.5 |

The value of S50 interpolated by second order polynomial regression analysis for Example 4 is 82 percent.

### Example 5

The procedure of this example is identical to Example 1, except that the active phase impregnation solution contained 29.5 g Fe(III) nitrate nonahydrate, 12.5 g potassium nitrate, and 0.46 g ammonium tungstate. After calcination, a red-orange powder was produced with a theoretical active phase concentration (wt/wt) of 3 percent Fe₂O₃, 3 percent K₂O, and 0.2 percent WO₃. The catalyst packed, bulk density was 1.09 g/cc. The conversion/selectivity data are given in Table 5.

TABLE 5

| Temperature ° C. | Percent Conversion | Percent Selectivity to Styrene |
|---|---|---|
| 547.5 | 37.9 | 87.6 |
| 573.1 | 45.4 | 83.8 |
| 595.3 | 60.7 | 77.6 |

The value of S50 interpolated by second order polynomial regression analysis for Example 5 is 82 percent.

### Example 6

The iron, potassium, indium catalyst from Example 1 was repeated and evaluated again, except that the diluent gas used was ethane instead of nitrogen. The procedure was otherwise the same as Example 1. During the run, the ethane was dehydrogenated to ethylene concurrently, but to a lesser extent than ethylbenzene and had conversions ranging from 0.5-15 percent. The data on conversion/selectivity is given in Table 6.

TABLE 6

| | Percent Conversion | |
|---|---|---|
| Temperature ° C. | of Ethylbenzene | Percent Selectivity to Styrene |
| 584.9 | 42.1 | 87.0 |
| 585.6 | 44.1 | 87.0 |
| 604.5 | 52.5 | 82.2 |
| 603.5 | 56.6 | 81.3 |
| 603.6 | 56.0 | 83.0 |

The value of S50 interpolated by second order polynomial regression analysis for Example 6 is 84 percent.

### Example 7

The iron, potassium and cerium catalyst of Example 2 was reloaded and evaluated again using an ethane diluent instead of nitrogen as before. The procedure was otherwise identical. The ethane was dehydrogenated to ethylene, but to a lesser extent than ethylbenzene and conversions ranged from 0.5-15 percent. The data are shown in Table 7.

TABLE 7

| | Percent Conversion | |
|---|---|---|
| Temperature ° C. | of Ethylbenzene | Percent Selectivity to Styrene |
| 586.8 | 50.8 | 84.1 |
| 587.4 | 51.5 | 82.3 |
| 603.6 | 60.3 | 75.0 |
| 604.9 | 63.2 | 72.5 |

The value of S50 interpolated by second order polynomial regression analysis for Example 7 is 84 percent.

### Example 8

The procedure of this example is identical to Example 1, except that the catalyst composition was 3 percent Fe₂O₃, 3 percent K₂O and 0.11 percent CaO. The data show that the value of S50 interpolated by second order polynomial regression analysis for Example 8 was 84 percent.

### Example 9

The procedure of this example is identical to Example 1, except that the catalyst composition was 3 percent Fe₂O₃, 3 percent K₂O and 0.33 percent Sm₂O₃. The data show that the value of S50 interpolated by second order polynomial regression analysis for Example 9 was 84 percent.

### Example 10

The procedure of this example is identical to Example 1, except that the catalyst composition was 3 percent Fe₂O₃, 3 percent K₂O and 0.16 percent ZnO. The data show that the value of S50 interpolated by second order polynomial regression analysis for Example 10 was 81 percent.

### Example 11

The procedure of this example is identical to Example 1, except that the catalyst composition was 3 percent Fe₂O₃, 3 percent K₂O and 0.18 percent Mn2O₅. The data show that the value of S50 interpolated by second order polynomial regression analysis for Example 11 was 81 percent.

### Example 12

The procedure of this example is identical to Example 1, except that the catalyst composition was 3 percent Fe₂O₃, 3 percent K₂O and 0.15 percent CuO. The data show that the value of S50 interpolated by second order polynomial regression analysis for Example 12 was 80 percent.

### Example 13

The procedure of this example is identical to Example 1, except that the catalyst composition was 3 percent Fe₂O₃, 3 percent K₂O and 0.31 percent La₂O₃. The data show that the value of S50 interpolated by second order polynomial regression analysis for Example 13 was 79 percent.

When the concentration of selectivity improvers is decreased the selectivity also decreases, as shown in the following examples.

### Examples 14 and 15

14. The procedure of Example 2 was repeated, except that the final catalyst had a concentration of 0.33 percent CeO₂. The selectivity value S50 of the conversion to styrene decreased to 72 percent.

15. The procedure of Example 4 was repeated, except that the final catalyst had a concentration of 0.28 percent MoO₃. The selectivity value of S50 decreased to 80 percent.

In some cases it has been found that certain metals at specific concentrations are less effective in enhancing the selectivity of the conversion of, for example, ethylbenzene to styrene. In an example which is not representative of the invention, cobalt oxide, as 0.16 percent Co₂O₃, was used in an experiment in the same manner as Example 1, but had an S50 value of 71 percent.

### Comparative Example

The procedure is identical to Example 1 except for the following changes in the active phase composition of the catalyst. The active phase impregnation solution contained 29.5 g Fe(III) nitrate nonahydrate, 12.5 g potassium nitrate. After calcination, a red-orange powder was produced with a theoretical active phase concentration (wt/wt) of 3 percent Fe₂O₃, and 3 percent K₂O. The catalyst packed, bulk density was 1.02 g/cc. The reference/comparative catalyst conversion/selectivity data are given in Table 8.

TABLE 8

| Temperature ° C. | Percent Conversion | Percent Selectivity to Styrene |
|---|---|---|
| 550.3 | 29.8 | 89.9 |
| 573.7 | 43.7 | 81.1 |
| 597.2 | 53.6 | 79.2 |
| 599.8 | 57.3 | 77.9 |

The value of S50 interpolated by second order polynomial regression analysis for the Comparative Example is 80 percent.

The catalysts of Examples 1-11 show improved selectivity to styrene (S50) compared with the Comparative Example containing only iron and potassium. The catalysts of Examples 12 and 15 have the same selectivity as the Comparative Example, even though one sample is at a lower concentration than another experiment and Example 13 is just slightly lower than the Comparative Example. Further, the improved selectivity is maintained after a change in the diluent gas from nitrogen to ethane, in which the ethane itself was dehydrogenated to ethylene.

From the foregoing Examples it is clear that another aspect of the present invention resides in a process for producing a dehydrogenated alkyl aromatic hydrocarbon compound. Preferably, the process of dehydrogenating an alkyl aromatic hydrocarbon compound with the calcined dehydrogenation catalyst described hereinabove, optionally in the presence of a diluent gas, at a temperature sufficient to effect the dehydrogenation to a vinyl aromatic hydrocarbon compound. Preferably, the alkyl aromatic compound is ethylbenzene, isopropylbenzene, or alpha-methyl ethylbenzene. Although not covered by the term alkyl aromatic hydrocarbon compound, the present process may also be used to convert unsaturated alkenyl compounds to di-unsaturated compounds, such as, converting butylene to butadiene. In similar fashion, other unsaturated compounds can be envisioned to likewise be converted into the di-unsaturated compounds. Further, paraffinic hydrocarbon compounds can be converted to unsaturated compounds; for example, ethane can be converted into ethylene. Likewise, depending on the feedstream, several of such compounds can be converted to their unsaturated counterparts in the same process at the same time.

The process of the present invention can be carried out in a fixed bed, fluid-bed with reactor-regenerator system, or other convenient reactor system. It has been found that a fluid bed reactor-regenerator system affords good heat management, adequate contact and satisfactory yields of vinyl aromatic hydrocarbon compounds. A suitable fluid bed reactor-regenerator system has been described in PCT WO 0123336 A1 20010405 by Iezzi and Sanfilippo, which is hereby incorporated by reference as if fully set forth. Using this reactor-regenerator system, an alkyl aromatic hydrocarbon compound, which may be preheated to from 200 to 400° C., is introduced into the reactor which is operated at from 500 to 600° C. in countercurrent flow with the circulation of the calcined dehydrogenation catalyst of this invention. The reactor effluent gas is separated overhead from the catalyst particles by appropriate means and the effluent gas stream is purified to obtain the product vinyl aromatic hydrocarbon compound. The remainder of the effluent gas is then either recycled or used as fuel gas to supply, for example, preheat to the feedstream. The catalyst is separated from the reactor and transferred to the regenerator where an oxygen-containing gas is employed to convert any residual hydrocarbon to gas and regenerate the calcined dehydrogenation catalyst. The regenerated catalyst, after separation from the regenerator, is then returned to the reactor.

1. A calcined dehydrogenation catalyst comprising a calcination product of
a) at least one iron oxide or a carbonate, bicarbonate, nitrate, hydroxide, oxalate or other similar conjugate base of a weak acid;
b) a carbonate, bicarbonate, nitrate, hydroxide, oxide or oxalate of an alkali metal or other similar conjugate base of a weak acid;
c) a carbonate, bicarbonate, nitrate, hydroxide, oxide or oxalate or other similar conjugate of a weak acid of at least one member of the group consisting of indium, calcium, samarium, cerium, sodium, molybdenum, tungsten, zinc, manganese, copper and lanthanum; and
d) an alumina or silica-alumina support material having a bulk density from 0.9 to 1.3 grams per cubic centimeter, and an average particle size of from 30 to 300 microns.

2. The calcined dehydrogenation catalyst of claim 1 wherein (c) is indium.

3. The calcined dehydrogenation catalyst of claim 1 wherein (c) is cerium

4. The calcined dehydrogenation catalyst of claim 1 wherein (c) is sodium.

5. The calcined dehydrogenation catalyst of claim 1 wherein (c) is calcium.

6. The calcined dehydrogenation catalyst of claim 1 wherein (c) is samarium.

7. The calcined dehydrogenation catalyst of claim 1 wherein (c) is tungsten.

8. The calcined dehydrogenation catalyst of claim 1 wherein (c) is molybdenum.

9. The calcined dehydrogenation catalyst of claim 1 wherein (c) is present in an amount of from 0.01 to 4 percent by weight based on the weight of the total catalyst composition.

10. The calcined dehydrogenation catalyst of claim 1 wherein in (b) the alkali metal is potassium in the form of the oxide and wherein (c) is indium oxide.

11. The calcined dehydrogenation catalyst of claim 9 wherein (c) is cerium oxide.

12. The calcined dehydrogenation catalyst of claim 9 wherein (c) is calcium oxide.

13. The calcined dehydrogenation catalyst of claim 9 wherein (c) is samarium oxide.

14. The calcined dehydrogenation catalyst of claim 9 wherein (c) is sodium oxide.

15. The calcined dehydrogenation catalyst of claim 9 wherein (c) is molybdenum oxide.

16. The calcined dehydrogenation catalyst of claim 1 wherein the bulk density is from 0.95 to 1.1 grams per cubic centimeter.

17. A process for preparing a calcined dehydrogenation catalyst comprising:
a) adding an active phase in the form of an aqueous solution of
(i) at least one iron oxide oxide or a carbonate, bicarbonate, nitrate, hydroxide, oxalate or other similar conjugate base of a weak acid;
(ii) a carbonate, bicarbonate, nitrate, hydroxide, oxide or oxalate or other similar conjugate base of a weak acid of an alkali metal; and
(iii) a carbonate, bicarbonate, nitrate, hydroxide, oxide or oxalate or other similar conjugate base of a weak acid of at least one member of the group consisting of indium, calcium, samarium, cerium, sodium, molybdenum, tungsten, zinc, manganese, copper and lanthanum to an alumina or silica-alumina support material having a bulk density from 0.9 to 1.3 grams per cubic centimeter and an average particle size of from 30 to 300 microns;
b) drying the support material containing the active phase to remove the water; and
c) calcining the dried support material containing the active phase to a finished catalyst.

18. The process of claim 17 wherein (a)(iii) is indium.

19. The process of claim 17 wherein (a)(iii) is cerium.

20. The process of claim 17 wherein (a)(iii) is sodium.

21. The process of claim 17 wherein (a)(iii) is molybdenum.

22. The process of claim 17 wherein (a)(iii) is tungsten.

23. The process of claim 17 wherein (a)(iii) is calcium.

24. The process of claim 17 wherein (a)(iii) is samarium.

25. The process of claim 17 wherein (a)(iii) is an oxide which is present at from 0.01 to 4 weight percent based on the total weight of the finished catalyst composition.

26. The process of claim 17 wherein in (b) the drying occurs at 80 to 200° C. for 1 to 12 hours and in (c) the calcining occurs at 500 to 950° C. for 3 to 8 hours.

27. The process of claim 17 wherein the bulk density of the finished catalyst is from 0.95 to 1.1 grams per cubic centimeter.

28. A process of dehydrogenating an alkyl aromatic hydrocarbon compound which comprises contacting said compound with the calcined dehydrogenation catalyst of claim 1 in the presence of a diluent at a sufficient temperature to dehydrogenate the alkyl aromatic hydrocarbon compound and produce a vinyl aromatic hydrocarbon compound.

29. The process of claim 28 in which the alky aromatic hydrocarbon compound is selected from ethylbenzene, isopropylbenzene and alpha-methyl ethlybenzene to produce styrene, cumene and alpha-methyl styrene, respectively.

30. The process of claim 28 in which said catalyst is separated from the contacting step for regeneration in an oxygen-containing gas, optionally in the presence of a diluent, so that any residual hydrocarbon is removed and the calcined dehydrogenation catalyst is restored to its original condition and recycled to said contacting step.

31. The process of claim 28 in which said diluent is a paraffinic hydrocarbon compound and an alkenyl hydrocarbon compound is also produced.
